(19) United States
(12) Patent Application Publication
(10) Pub. No.: US 2025/0020596 A1
(43) Pub. Date: Jan. 16, 2025

(54) DEVICES AND METHODS INVOLVING METADEVICES AND PHOTONIC-BASED BIOSENSING

(71) Applicant: The Board of Trustees of the Leland Stanford Junior University, Stanford, CA (US)

(72) Inventors: Jack Hu, Stanford, CA (US); Jennifer A. Dionne, Menlo Park, CA (US); Fareeha Safir, Menlo Park, CA (US); Butrus T. Khuri-Yakub, Stanford, CA (US)

(21) Appl. No.: 18/712,142

(22) PCT Filed: Nov. 23, 2022
(86) PCT No.: PCT/US2022/050970

§ 371 Date: May 21, 2024

Related U.S. Application Data
(60) Provisional application No. 63/283,037, filed on Nov. 24, 2021.

(51) Int. Cl. G01N 21/77; G01N 33/543

(52) U.S. Cl. CPC G01N 21/77; G01N 33/54373; G01N 2021/7753; G01N 2021/7789

(57) ABSTRACT

In certain examples, methods and semiconductor structures, aspects of the disclosure are directed to a guided-mode resonance metasurface pixel (“GMR pixel”) having a cavity section to support GMR at a certain Q and having optics at each end of the GMR pixel, to contain light and mitigate energy losses due to scattering of light, in response to light being directed towards the GMR pixel of the metasurface sensor. In certain more-specific examples, exemplary aspects of the disclosure are directed to a functionalized metasurface sensor including an array of guided-mode resonance metasurface biosensor pixels, each of which is functionalized for attachment of a distinct receptor or probe molecules.

### BACKGROUND

In certain exemplary contexts, aspects of the present disclosure are directed to apparatuses and methods involving guided-mode resonance metasurface pixel structures and designs having aspects to support GMR at a certain quality factor and involving analysis of biomolecules and chemical species associated with the genome, proteome, transcriptome, epigenome, metabolome, and/or microbiome. Such analysis is important for understanding, monitoring, and treating organism and ecosystem health. Integration of data from multiple biomarkers, such as both nucleic acids and proteins, from a single sample or specimen provides deeper insight into complex biological processes or geno-pheno-envirotype relationships than analysis of individual biomarkers alone.

For such analysis, providing apparatuses and/or methods in efficient, cost-effective, and convenient manners has been difficult. Moreover, attempts to analyze multitudes and/or multiple classes of biomarkers in such manners has been complex and burdensome. These and other matters have presented challenges, for a variety of applications including those in clinical and research settings.

### SUMMARY OF VARIOUS ASPECTS AND EXAMPLES

Various examples/embodiments presented by the present disclosure are directed to issues such as those addressed above and/or others which may become apparent from the following disclosure. For example, some of these disclosed aspects are directed to methods and devices that use or leverage from mitigation or an optimization of energy. In certain examples, for detecting distinct aspects of the sample, manipulated by a biological sample and then processed by a distinct receptor or probe molecules.

In certain examples, methods and apparatuses (e.g., electronic devices) are directed to a guided-mode resonance metasurface pixel (“GMR pixel”) having a cavity section to support GMR at a certain Q and having optics at each end of the GMR pixel, to contain light and mitigate energy losses due to scattering of light, in response to light being directed towards the GMR pixel of the metasurface sensor. In certain more-specific examples, exemplary aspects of the disclosure are directed to a functionalized metasurface sensor including an array of guided-mode resonance metasurface biosensor pixels, each of which is functionalized for attachment of a distinct receptor or probe molecules.

In other more-specific example aspects of the present disclosure (which may relate to or build upon one or more of the above methods and apparatus), such a functionalized metasurface sensor may include an array of high-Q, guided-mode resonance metasurface biosensor pixels (“GMR pixels”), each of which is functionalized for attachment of a set of at least one distinct receptor and/or at least one probe molecule. Each of the GMR pixels, which may be energized by the directed light, may have optics on either side of the cavity section so that the optics can act as photonics mirrors and prevent leakage of energy and reflect the energy back towards the cavity section.

In connection with further more-specific aspects which may relate to or build on the above aspects, the present disclosure is directed to: a partially—or completely—manufactured apparatus as in the exemplary aspects and embodiments as described or characterized herein: a method of using (e.g., testing, measuring, and/or in research/clinical settings) such an apparatus as described or characterized herein; and a method of making a partially—or completely—manufactured apparatus as characterized or described herein.

In more specific examples which may also build on the examples discussed herein, aspect are directed to one or more (in combination) of the following: blocks of the GMR pixel are to react to light directed towards a surface of the GMR pixel, and to cause formation of a photonic bandgap, where no photonic modes with frequencies within the bandgap are allowed to propagate, and wherein frequencies corresponding to the edge of the bandgap are tuned based on the dimensions of the nanoblocks: the optics include, at each end of the GMR pixel and on opposing sides of the cavity section, a first set of one or more nanoblocks and a second set of one more nanoblocks: the optics function or act as photonic mirrors by containing a resonant mode: the cavity section has multiple nanoblocks of different dimensions, one of the different dimensions being distinguishable from another of the different dimensions in terms of nanoblock length: wherein the cavity section has multiple nanoblocks to support GMR at a certain high-Q characterized depending on the design and application (e.g., ranging from anywhere of Q being greater than 10 and in many instances and applications, the high Q is in the thousands or tens of thousands), the cavity section has multiple nanoblocks of different lengths which are dimensioned such that the respective ends of the multiple nanoblocks collectively align to form a tapered dimension of at least a portion of the cavity section between the optics ends (e.g., as shown in FIG. 6); the cavity section has multiple nanoblocks of different dimensions, one of the different dimensions being distinguishable from another of the different dimensions in terms of nanoblock length; and at least one of the GMR pixels has a material capable of being energized by the directed light and has at least one section with a length corresponding to an illumination wavelength energy that is below a band gap of the material.

In one specific example, the present disclosure is directed to a method having certain of the above aspects disclosed hereinabove for an apparatus. For example, one such method according to the present disclosure includes steps of functionalizing a metasurface sensor, and/or securing optics at respective ends of each of a plurality of the GMR pixels. The step of functionalizing a metasurface sensor concerns a metasurface sensor characterized as including an array of (e.g., high-Q) guided-mode resonance metasurface biosensor pixels (“GMR pixels”), by attaching a distinct receptor or probe molecules to respective ones of the GMR pixels. The step of securing optics at respective ends of each of a plurality of the GMR pixels, is to contain light and optimally mitigate energy losses due to scattering of light, in response to light being directed towards the metasurface sensor. In this above approach, the circuitry and the functionalization permit the directed light, after being manipulated by a biological sample, so that different types of biological molecules in the biological sample can be distinguished at the GMR pixels.

In more specific embodiments, the above method may further include: distinguishing the different types of biological molecules in the biological sample; and/or using the circuitry to direct the light towards the metasurface sensor, wherein the circuitry includes a CCD and a logic circuit to respond to the directed light.

In yet further related embodiments applicable to the above type of method, the method may include patterning the distinct receptor or probe molecules to respective ones of the GMR pixels through acoustic droplet ejection, and/or may include customizing or matching surface functionalization resolution of individual ones of the GMR pixels to detect distinct biomarkers relative to neighboring ones of the GMR pixels or certain distinct target species of samples for analysis.

In yet a further example also related to the above type of method, the distinct receptor or probe molecules may be attached to surfaces of the GMR pixels by applying a material to minimize nonspecific adsorption for binding to an antibody of interest.

In more specific examples related to the above methodology and/or apparatus, an electronic device (e.g., as part of such an apparatus, may include the distinct receptor or probe molecules for attachment to respective ones of the metasurface biosensor pixels.

According to other related aspect which may also build on one or more of the above examples and aspects, the present disclosure is directed to respective ones of the metasurface biosensor pixels being are bio printed.

According to yet another related aspect, the present disclosure is directed to one or more aesthetic—or ornamental—design aspects characterizing one or more of the structures shown in the figures herein (e.g., the pixel structures and/or the metasurface array).

The above discussion is not intended to describe each aspect, embodiment or every implementation of the present disclosure. The figures and detailed description that follow also exemplify various embodiments.

### BRIEF DESCRIPTION OF FIGURES

Various example embodiments, including experimental examples, may be more completely understood in consideration of the following detailed description in connection with the accompanying drawings, each in accordance with the present disclosure, in which:

FIG. 1 is a graph, in accordance with the present disclosure, depicting calculated Q factors as a function of the structural perturbation Δd for an infinitely long resonator (linear solid (black) line), for a waveguide device truncated to only 10 μm (curved solid (blue) line), and wherein the total quality factor is limited by the maximum quality factor associated with scattering losses (horizontal (red) dashed line):

FIGS. 2A and 2B are related graphs, in accordance with the present disclosure, relating to changes in lengths of the silicon nanoblocks along the waveguide to effect shifts of the bandgap towards different (e.g., lower) frequencies:

FIG. 3A shows an example sensing pixel design and a related graph of Q factor versus Δd (nanometers), for infinitely long also in accordance with the present disclosure, with the pixel design showing a cavity section with two different lengths of blocks to support GMR at a certain high Q and with an optics section at each end including at least one (e.g., five) blocks of similar length (length being the horizontal dimension);

FIG. 3B shows the example sensing pixel design of FIG. 3A from a perspective view showing a substrate structure, with an upper surface which the blocks of at least one pixel is secured, also according to the present disclosure:

FIG. 4 (in connection with a dual set of related graphs and a formula for computing mirror strength also in accordance with the present disclosure) shows how use of such exemplary sensing pixel design types may allow for up to an order of magnitude increase in Q compared to devices that have no mirror elements to contain the guided mode resonance within the waveguide:

FIG. 5 shows exemplary pixel designs (with size dimensions in microns along the X-axis and Y-axis) to help visualize such a magnitude increase in Q (from ˜8000 to ˜80,000), also in accordance with certain specific examples of the present disclosure:

FIG. 6 shows exemplary scanning electron micrographs of other similarly constructed example experimental high-Q pixel structures (e.g., with one being ˜10 μm in length and having portions of the cavity section with blocks collectively aligned to form a tapered dimension and with blocks at the ends of the one pixel to provide optics for reflecting light), also in accordance with certain specific examples of the present disclosure:

FIG. 7A shows how the spectral response of multiple pixels (such as depicted in the example of FIG. 6) can be measured simultaneously through the intensity at each pixel location;

FIG. 7B shows an image corresponding to multiplexed metasurface(s) being functionalized using droplet generation or chemical transfer for surface functionalization at the resolution of an exemplary patterned metasurface; and

FIG. 8 is a perspective view of a sensor system including an array of GMR pixels, also according to the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of apparatuses, systems and methods involving optical metastructures and/or metasurfaces (sometimes collectively referred to as “metadevices”) having nanostructured silicon blocks and/or films and which may have a number of applications depending on how they are designed. According to one specific example aspect of the present disclosure, such optical metadevices are to detect biomarker surface binding by way of their respective metasurfaces resonantly trapping light along patterned silicon nanostructures, and/or involving guided mode resonances (GMR) supported by a cavity section of each pixel designed for the metadevice. This specific aspect for biomarker surface binding exemplifies one from among other specific aspects, according to the present disclosure, which may be benefited by one or more metadevices having multiple nanostructures thereon to function or act, collectively, as a set of photonic mirrors that contain a resonant mode, associated with one or more GMR for light manipulated by the metadevice. This type of design is especially advantageous for a relatively short (or shortened) sensor device. While the following discussion refers to such aspects as exemplified in certain apparatuses and methods, such discussion is for merely providing exemplary contexts to help explain one or more of such aspects, and the present disclosure is not necessarily so limited.

According to certain specific example aspects, in which such optical metadevices are used for biomarker surface binding according to the present disclosure, by using one or more metadevices having multiple nanostructures to support a high-Q GMR for light manipulated by a sample, this type of design is especially advantageous for a relatively short (or shortened) sensor device because without significant loss in the high-Q, losses due to scattering from waveguide ends are minimized. In more specific structures, multiple nanostructures (or blocks) are utilized at the ends of each pixel to act as photonic mirrors, thereby reflecting the light back towards the center (cavity portion) and containing the high-Q resonant mode within a relatively short sensor device.

In certain specific example, due to the significant refractive index contrast between the silicon nanostructures and the surrounding medium, there is significant Bragg scattering along the waveguide device. This leads to the formation of a photonic bandgap, where no photonic modes with frequencies within the bandgap are allowed to propagate. In certain more specific aspects of the present disclosure, the frequencies corresponding to the edge of the bandgap can be tuned by changing the dimensions of the blocks along the waveguide chain.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same connotation and/or reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

Further, certain aspects of the present disclosure are directed not only to a completely-manufactured apparatus as in the exemplary aspects and embodiments as may be described or characterized in certain portions of the present disclosure, but also to aspects of partially-manufactured apparatus and/or elements of the apparatus (e.g., the optics or photonic mirrors, high-Q pixel structures, metasurface array, etc.) which are to be assembled and included with a more complete apparatus consistent with certain exemplary aspects and embodiments described or characterized herein. Also, certain other aspects of the present disclosure are directed methods of using (e.g., testing, measuring, and/or in research/clinical settings) such an apparatus (or its elements).

Exemplary aspects of the present disclosure are directed to methods and devices involving an optimization of energy, for detecting distinct aspects of the sample, manipulated by a biological sample and then processed by a distinct receptor or probe molecules. In certain examples, methods and semiconductor structures are directed to a functionalized metasurface sensor including an array of high-Q, guided-mode resonance metasurface biosensor pixels (“GMR pixels”), each of which is functionalized for attachment of at least one distinct receptor or probe molecule. Optics are coupled at respective ends of each of the GMR pixels, to contain sample-manipulated light (by a biological sample) and optimally mitigate energy losses due to scattering of light and further, to distinguish between different types of biological molecules in the biological sample, light-responsive and data-processing circuitry responds to the sample-manipulated light at respective ones of the distinct receptor or probe molecules. One or more of the GMR pixels may have a material capable of being energized by the directed light and has at least one section with a length corresponding to an illumination wavelength energy that is below a band gap of the material, and at least one of the GMR pixels may have the optics including a cavity section and respective sets of one or more blocks to act as respective photonics mirrors on either side of the cavity section (e.g., to mitigate or prevent lost energy due to scattering of the light by directing light energy towards the cavity section).

Aspects of various embodiments are directed to apparatuses (e.g., systems, devices, assemblies, components, materials, etc.), and any of various types of processes (e.g., methods of use and methods of manufacture) involving one or more of such apparatuses such as those described in the claims, description and figures. For information regarding details of other embodiments, experiments and applications that can be combined in varying degrees with the teachings herein, reference may be made to the teachings and underlying references disclosed in U.S. Provisional Application Ser. No. 63/283,037 filed on Nov. 24, 2021 (STFD.438P1 S21-418) with Appendices, to which priority is claimed (and to the extent permitted, such subject matter is incorporated by reference in its entirety and specifically for further examples and more-detailed embodiments as may be useful to supplement and/or clarify).

In certain more specific example embodiments, the present disclosure is directed to an optical platform for rapid, label-free, high throughput, and multiplexed biomarker analysis. This platform enables detection of multiple distinct DNA and RNA sequences, as well as different proteins, on a single chip and from a single sample, without relying on recognition any fluorescent or optical tagging. In certain more specific aspects, the present disclosure is directed to providing a compact, rapid, and potentially low cost platform for targeted DNA and RNA sequencing, where thousands of distinct nucleic acid probe sequences can be arrayed across the exemplary sensing device for multiplexed genetic and transcriptomic analysis. Additionally, proteins and metabolites can also be detected on the same platform, to enable multiomics datasets for determining geno-pheno-envirotype associations. Such aspects allow for big-data acquisition to improve early disease detection, treatment monitoring of cancer and infectious diseases, understanding of neurological disorder progression, and detection of toxins in agricultural or environmental settings.

In certain specific example embodiments, aspects of the present disclosure are directed to a method for use in a system in which a nanophotonic waveguide-based device manifests radiation losses, and wherein the device takes into account or mitigates reduction of Q factors associated with photons scattering from truncated ends of the waveguide. Certain more specific related embodiments are directed to an apparatus or method including or involving: high quality (high-Q) factor metasurfaces (e.g., where Q is at least several thousand, and in some instances greater than 1000 or greater than 10,000 (e.g., ˜12,000, ˜15,000, ˜20,000, or higher), for use with bio sensing samples such as in connection with detection of nucleic acids, proteins, pathogens, and small molecules). In another example, the high-Q is characterized as being in a range from 1000 up to 40,000 or 60,000. In such examples, one or more photonic mirror elements help to spatially confine the guided mode resonances (GMR) and/or prevent scattering losses: use of such photonic mirrors are used in connection with metasurface device dimensions that are truncated to produce compact and individually addressable biosensing pixels; and/or a design directed to the aesthetic or ornamental design aspects of one or more of the structures shown in the figures herein (e.g., one or more of the high-Q pixel structures in combination and/or an array of such structures as illustrated in connection with the metasurface).

In yet further specific aspects, the present disclosure is directed to one of the following as utilized or implemented as an individual aspect, or two or more of the following in combination (wherein each such aspect is exemplified without limitation with regards to other examples presented herein): (a) a high-Q pixel sensing element design with metasurface design features that enable truncation and shrink the resonant devices into relatively small footprints sufficient to enable individually-addressable sensing devices patterned at high densities and/or enable dense microarrays of sensing elements that may act as a platform for massively multiplexed and parallelized biomarker detection; (b) free space radiation control and high radiative Q factors; (c) photonic mirrors to mitigate loss of Q from end scattering; (d) fabrication and optical characterization of metasurface array; (e) metasurface functionalization and multiplexed functionalization methods; (f) multiplexed metasurface functionalization; (g) metasurface functionalization for protein detection; and (h) acoustic bioprinting of such metasurface-related aspects or designs.

In connection with more specific examples which are consistent with certain of the above aspects, such a manufactured device or method of such manufacture may involve bioprinting (e.g., acoustic bioprinting) of such metasurface-related aspects or designs. According to other related aspects, the present disclosure is directed to or may involve printing based surface functionalization of a chip surface. In connection with these aspects, bioprinting is utilized to attach distinct receptor molecules to individual metasurface biosensor pixels. The small droplet size of bioprinting allows for spatial patterning of arrays of sensing pixels such that individual pixels can be modified to detect a distinct biomarker from its neighboring pixel elements.

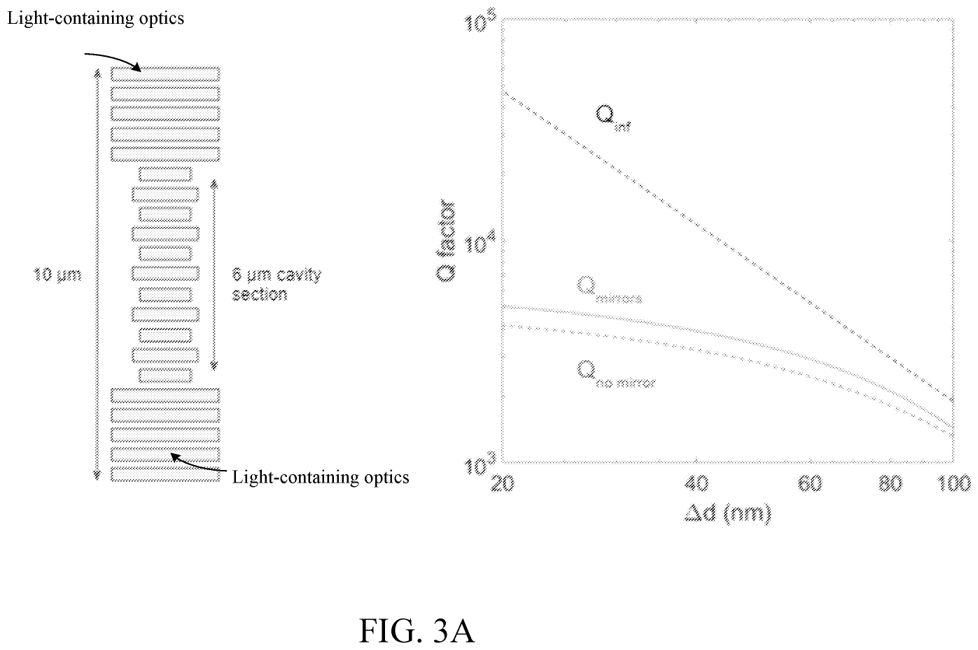

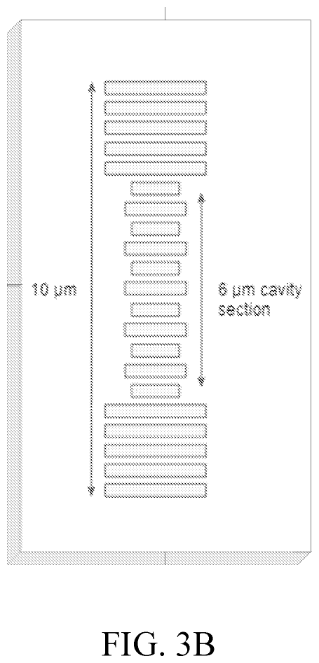

In certain specific example embodiments according to the present disclosure, an acoustic bioprinting method is used to provide chemical or biological functionalized groupings of biosensors or individual resonators with unique surface functionalizations. One form of acoustic printing uses a focused sound wave to eject droplets without a flow focusing nozzle. As such, the droplet diameter is a function of the acoustic transducer resonant frequency—the droplet diameter is inversely proportional to the resonant frequency. As such, droplet volumes can be varied by varying the resonant frequency of the printer. In further certain experimental proof-of-concept efforts in connection with the present disclosure, the instant inventors have demonstrated stable biological printing with 4 resonant frequencies varying between 5 MHz and 147 MHz generating droplets between 300 μm and 15 μm in diameter or 4.5 nL to 2 pL in volume, respectively (as demonstrated in slide 1 of the attached slides). The printer may be used to print and deposit an array of biological functionalizations onto chips (such as shown in FIGS. 3B, 6 and 8). The printer can be used with different chemistries, and can be used to deposit antibodies onto an array of GMRs/resonators and measure the resonant frequency shift with the binding of this probe antibody along with a target protein (e.g., the receptor binding domain of the COVID spike protein) that was incubated on the chip. IN connection with these efforts it has also been demonstrated that such a printer can be used to deposit 2 picoliter droplets onto single biosensor pixels, again using fluorescently tagged proteins.

Consistent with the present disclosure, such devices and/or methods may be used for producing (among other examples disclosed herein) such an apparatus as characterized above, with the functionalized metasurface sensor, the optics and the light-responsive and data-processing circuitry being configured cooperatively to enable detection of multiple distinct DNA and RNA sequences, as well as different proteins, on a single chip and from a single sample, without relying on recognition any fluorescent or optical tagging. Further, the distinct receptor or probe molecules may be configured for attachment and, in certain implementations, attached to respective ones of the metasurface biosensor pixels. In specific applications, such an apparatus may be used to detect and distinguish different types of biological molecules including one or more of: nucleic acids, proteins, pathogens, and small molecules.

Certain specific aspects of the present disclosure are directed to or involve high-Q-sensing element design which utilizes optical metasurfaces including or composed of nanostructured silicon films to detect biomarker surface binding. These metasurfaces resonantly trap light along the patterned silicon nanostructures, leading to strong electromagnetic field concentrations and sharp scattering spectra due to GMR. The GMR is excited via free space illumination from a laser or light emitting diode, and the scattered light is directed at a camera sensor. According to certain metasurface design features developed in manners consistent with the present disclosure, such high-Q-sensing aspects truncate and shrink the resonant devices into smaller footprints, enabling individually addressable sensing devices patterned at densities exceeding 1 million elements per square cm. These design features enable dense microarrays of sensing elements and can act as a platform for massively multiplexed and parallelized biomarker detection.

According to more-specific aspects and related to the above, the present disclosure is directed to or may involve high Q diffractive optical metasurfaces or high quality factor metasurfaces for use in connection with various types of bio sensing including, as examples, direct detection of nucleic acids, proteins, pathogens, and small molecules. Such more-specific aspects may leverage from and/or involve use of photonic mirror elements to spatially confine the GMR and prevent scattering losses when the metasurface device dimensions are truncated to produce compact and individually addressable biosensing pixels. Further, when combined with conventional (e.g., CMOS-based) fabrication processes, metasurface sensing elements according to the present disclosure may be patterned at densities exceeding hundreds of elements per square cm and in some instances exceeding many (e.g., one hundred, several hundred, or one million) elements per square cm. Due to the sharp scattering from the high-Q resonance, optical tagging of target molecules is not required (but may be used), and instead molecular binding is sensitively detected through changes in the scattered light intensity from each pixel. The dense sensor arrays allow for rich data sets acquired from a single image.

Discussion now turns to various more-specific examples of the present disclosure which are presented with the figures to illustrate certain more-specific (e.g., experimental/proof-of-concept) aspects and embodiments. Accordingly, as largely characterized above, such examples of the present disclosure are directed to different types of apparatus which involve a guided-mode resonance metasurface pixel (“GMR pixel”) having a cavity section to support GMR at a certain Q and having optics at each end of the GMR pixel, to contain light and mitigate energy losses due to scattering of light, in response to light being directed towards the GMR pixel of the metasurface sensor. A more-specific example of the present disclosure is directed to an apparatus having a high-Q pixel sensing element design with metasurface design features that enable truncation and shrink the resonant devices into smaller footprints, enabling individually addressable sensing devices patterned at high densities (e.g., one hundred to over one million elements per square cm), and/or enable dense microarrays of sensing elements that may act as a platform for massively multiplexed and parallelized biomarker detection.

According to certain experimental metasurface designs, a chain of nanostructured blocks are patterned into a high-refractive index material such as Silicon, Silicon nitride, Gallium nitride, Titanium dioxide, and/or Hafnium dioxide. A one-dimensional chain of nanostructures may constitute a waveguide that supports guided modes that are localized along the length of the nanopatterned chain. Structural perturbations are included longitudinally along the waveguide device to allow for free space coupling in high Q guided mode resonances. Through the structural perturbations along the chain, direct control is provided for the quality factor associated with the radiation losses of the mode as photons coupled in the GMR and eventually re-radiate out into free space. However, the total quality factor of a photonic mode is considered as the combination of all relevant photon loss mechanisms in the nanophotonic device. In connection with the present disclosure, considering a waveguide device that is infinitely long (or much longer than the photonic mode propagation length) and is operating at a illumination wavelength energy below the bandgap of the material constituting the nanophotonic device (thus reducing material absorption to negligible rates or zero), then only radiation losses are considered as relevant to the sensor design and can be controlled through geometric perturbations as described above. However, as the length of the waveguide device is shortened to construct more compact sensing pixels, such as those required to create a highly dense microarray of individual sensing elements, Q factors associated with photons scattering from the truncated ends of the waveguide are considered. As the waveguide device length is reduced, the guided mode resonance will increasingly scatter out of the end boundaries of the waveguide, reducing the overall Q factor and performance of the sensor. Thus, additional design elements, not previously considered, may be used to facilitate high-Q compact sensing pixels.

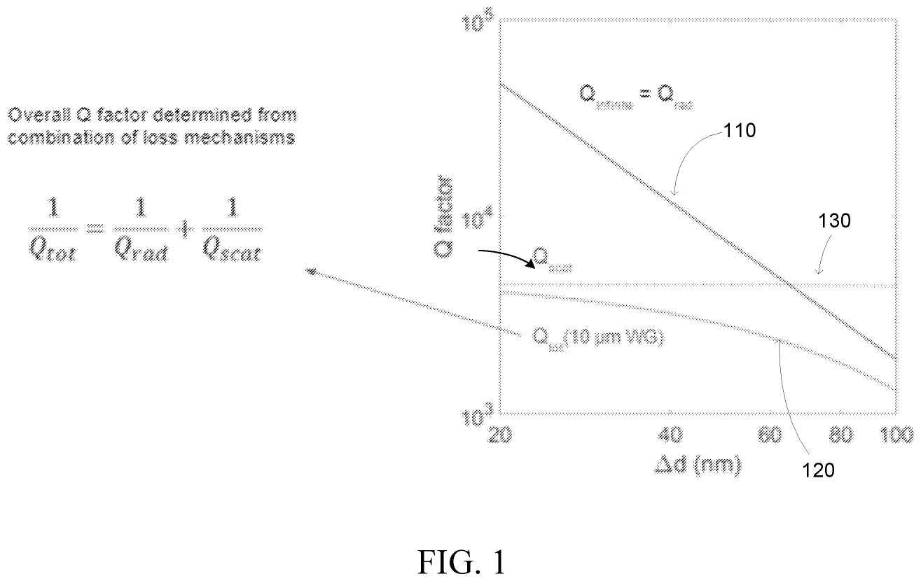

In FIG. 1, calculated Q factors are shown as a function of the structural perturbation Δd for an infinitely long resonator as indicated by the solid black line 110, and for a waveguide device truncated to only 10 μm as indicated by the solid blue line 120. Due to scattering from the truncated ends of the shortened structure, the total quality factor is limited by the maximum quality factor associated with scattering losses as indicated by the dashed line 130.

In order to minimize losses due to scattering from waveguide ends, additional nanostructures are utilized so as to act as photonic mirrors to contain the resonant mode within the shortened sensor device. Due to the significant refractive index contrast between the silicon nanostructures and the surrounding medium, there is significant Bragg scattering along the waveguide device. This leads to the formation of a photonic bandgap, where no photonic modes with frequencies within the bandgap are allowed to propagate. The frequencies corresponding to the edge of the bandgap can be tuned by changing the dimensions of the blocks along the waveguide chain.

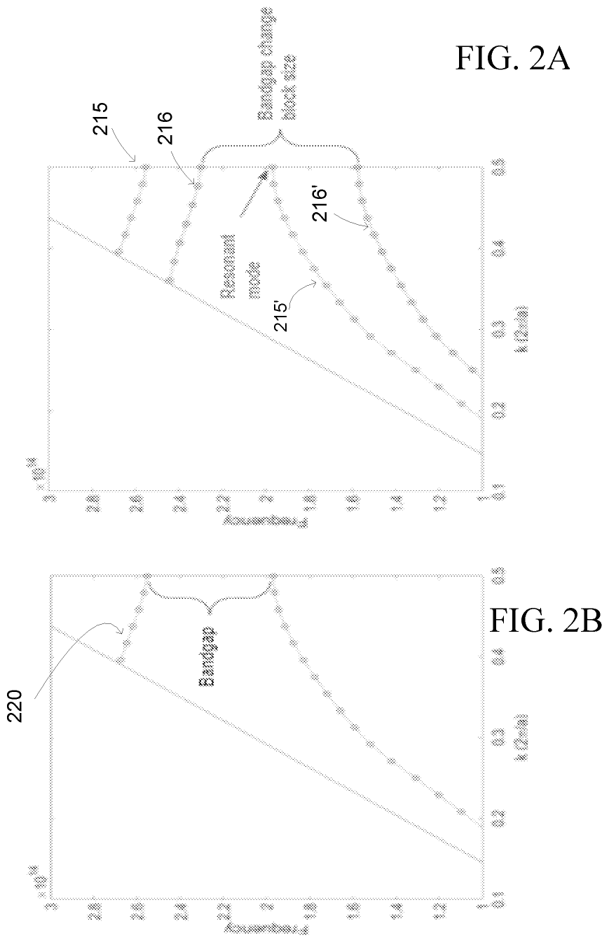

FIGS. 2A and 2B are related graphs, also according to aspects of the present disclosure, which show how exemplary silicon-nanoblocks on structures such as structures may be effectively increased in terms of the length of the silicon nanoblocks along the waveguide, so as to shift the bandgap towards lower frequencies. More specifically, FIGS. 2A and 2B show plots of frequency versus bandgap, for example experimental structures of different length dimensions, to depict operational aspects of a chain of silicon blocks with particular structural aspects including how dimensions can be altered to move the position of the bandstructure to different frequencies.

More particularly, the plots corresponding to the (blue) lines 215, 215′ and 220 of FIGS. 2A and 2B represent the band structure of a chain of silicon blocks corresponding with a particular structure (e.g., having height of 600 nm, width of 160 nm, length of 600 nm, and spacing of 330 nm). The absolute dimensions can be altered to move the position of the bandstructure to different frequencies. The (red) lines 216 and 216′ of FIG. 2A represent the photonic response of the photonic mirror like segments with longer blocks (as with the light-containing optics in the schematic of FIG. 3A, discussed below), and represent the bandstructure of a similar chain of silicon blocks, but with a length dimension for each block of ˜1300 nm (with all other dimensions fixed), leading to the shift in frequency from the blue line. Since the blue line 215′ of FIG. 2A has resonant modes at frequencies within the bandgap of the red line (216 and 216′) bandstructure, blocks of these two different dimensions (shorter 600 nm and longer 1300 nm) can be used to construct the high-Q pixel resonator as schematically depicted in FIG. 3A.

By designing a sensing pixel with sections with two different block lengths, according to the present disclosure a cavity section may be designed with a resonant mode corresponding to a band edge state, and a mirror section where the bandgap is shifted such that the mode frequency of the cavity section lies within the mirror bandgap. The mirror sections residing on either end of the resonator serve to prevent scattering of the guided mode resonance from the ends of the waveguide and instead reflect the mode back into the cavity section. Furthermore, the mirror strength can be systematically designed to optimally localize the photonic mode in the cavity by tapering the mirror segments to create a linearly varying mirror strength profile.

FIG. 3A shows examples of sensing pixel designs and a related graph of Q factor versus Δd (nanometers), with the Q factor for infinitely long chains composed of silicon blocks versus a shorter finite chain. As depicted by these plots of FIG. 3A, without use of a photonic mirror structure in accordance with examples of the present disclosure, the Q factor of the resonator device drops steeply as you reduce the physical size of the device. However, with the addition of such photonic mirror elements at the ends of the pixel device, high Q factors are maintained even for small devices (e.g., that are only ˜10-20 μm).

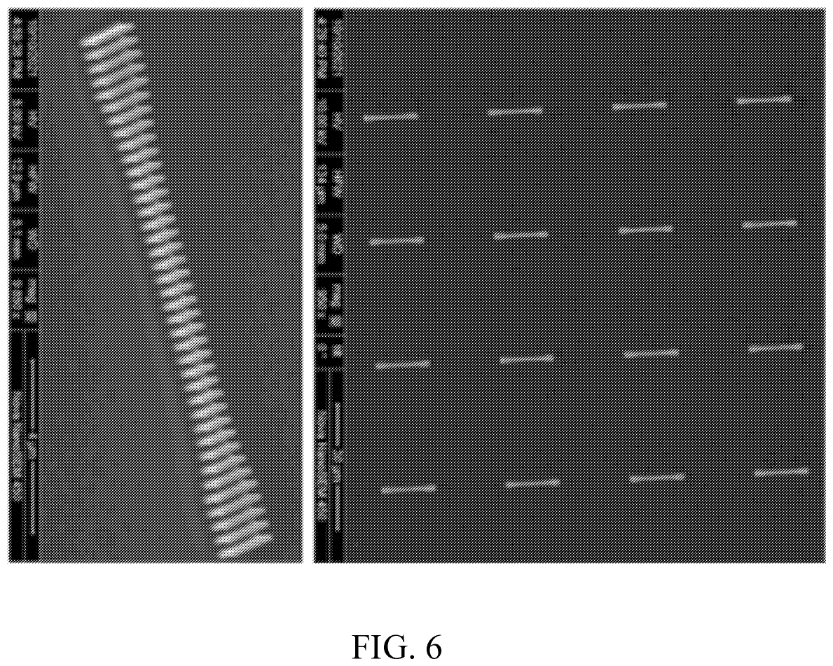

FIG. 3B shows the example sensing pixel design of FIG. 3A from a perspective view showing a substrate structure, with an upper surface which the blocks of at least one pixel is secured (and on which an array of pixels may be secured), a first longer side at the left (which may be identical in appearance to the opposite side), a second short side near the bottom of the sheet (which may be identical in appearance to the top opposite side). These illustration of the example sensing pixel design of FIG. 3A (and a similar structure as in the left and right portions of FIG. 6 but displaying a different surface showing different respective block lengths) are useful for showing aesthetic aspects of such designs according to the present disclosure.

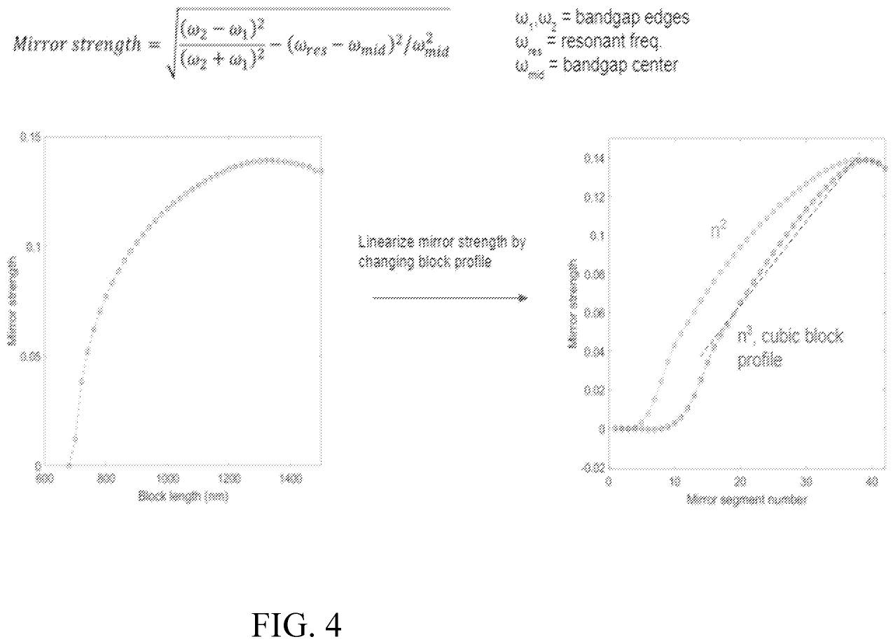

FIG. 4 (in connection with a dual set of related graphs and a formula for computing mirror strength also in accordance with the present disclosure) shows how use of such exemplary sensing pixel design types may allow for up to an order of magnitude increase in Q compared to devices that have no mirror elements to contain the guided mode resonance within the waveguide.

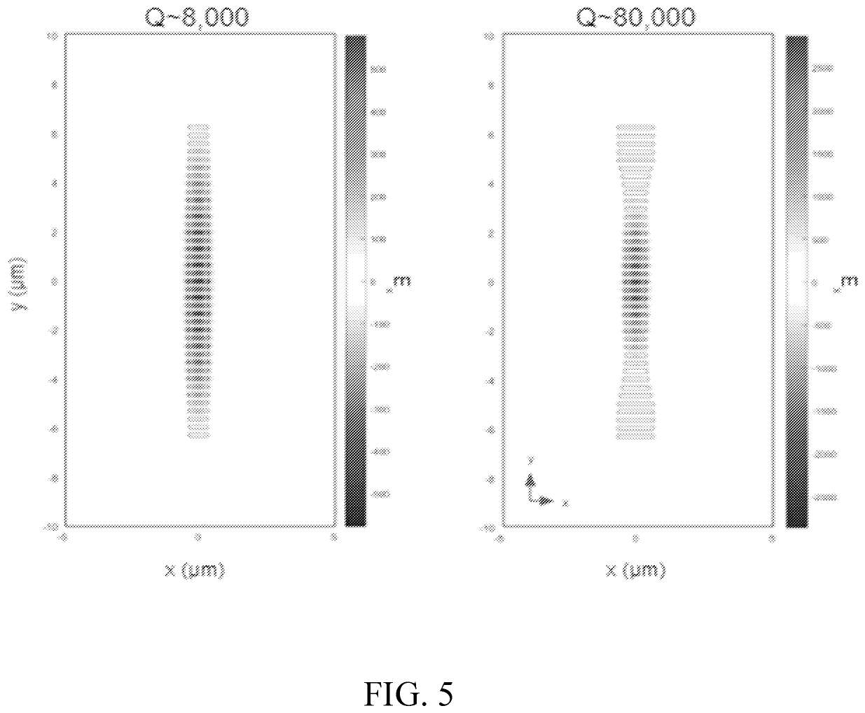

As illustrated by way of example in FIG. 5, according to this aspect design strategies allow for up to an order of magnitude increase in Q compared to devices that have no mirror elements to contain the guided mode resonance within the waveguide (the example scale on the right of each such device characterization of FIG. 5 spans from −2500 to 2500).

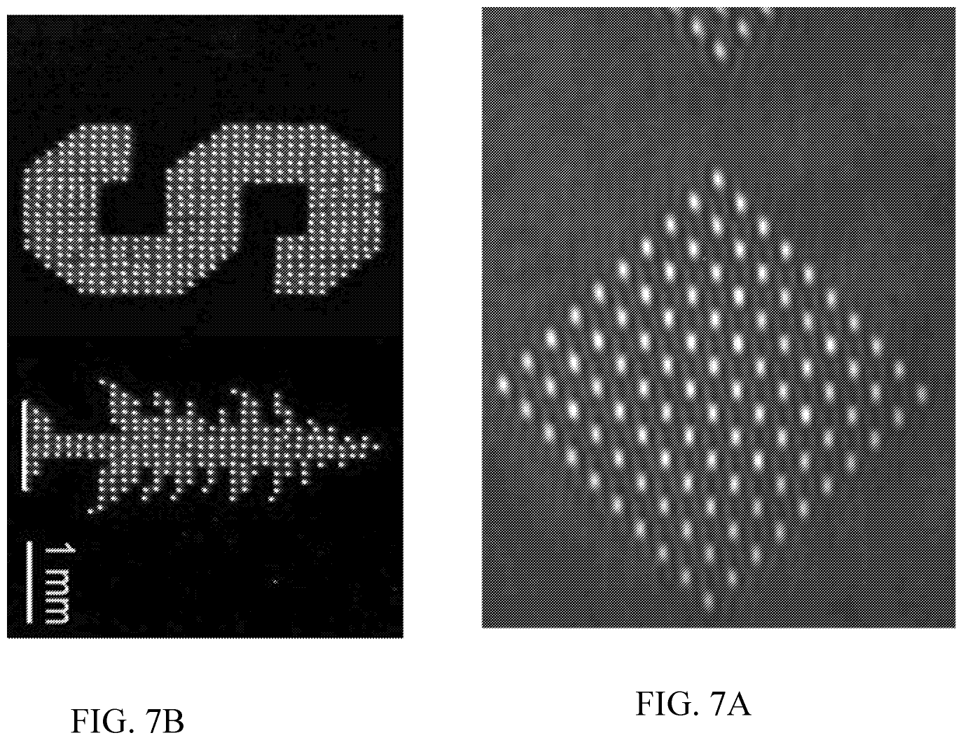

Metasurface pixels are fabricated in silicon films on a transparent sapphire substrate utilizing electron beam lithography. FIG. 6 shows scanning electron micrographs of fabricated high-Q pixel structures of 10 μm in length. Due to the spatial localization of the resonant mode, in such illustrative embodiments, each pixel only requires a footprint of 30 μm2. This compact footprint allows for pixel densities of up to ˜3 million features per square cm. When illuminated at normal incidence from free space, the nanostructures strongly scatter light in the transmitted and reflected directions towards a camera detector. The spectral response of multiple pixels can be measured simultaneously through the intensity at each pixel location as seen in FIG. 7A, where bright spots represent the scattered signal from an individual resonator.

Functionalization of exemplary metasurfaces allows for selective target binding and detection. In order to realize the multiplexed capabilities of such an exemplary device, the surface is functionalized to attach distinct receptor or probe molecules to individual metasurface biosensor pixels while simultaneously preventing nonspecific binding on the surfaces. Furthermore, utilization of a surface functionalization chemistry and patterning methodology is effected to allow functionalization of individual resonators with an array of probe molecules that allow for detection of nucleic acids, proteins, whole pathogens, and/or small molecules on a single chip. Surface chemical functionalization relies on covalent silanization of the metsurfaces with, for example, (3-aminopropyl) trimethoxysilane (APTMS) or 11-aminoundecyltriethoxysilane (AUTES). Amine-to-sulfydryl crosslinking with m-maleimidobenzoyl-N-hydroxysuccinimide (MBS) ester is then used to attach thiolated molecules necessary for probe binding. These initial steps can be completed in bulk across the entirety of a multiplexed chip.

Individual functionalization of each resonator for massively multiplexed biomolecular detection is effected. In one instance, the (integrated circuit) chips are utilized for 1) the multiplexed detection of numerous biomarkers from a single sample or 2) for the detection of a single biomarker from numerous samples.

For multiplexed detection of an array of biomarkers, picoliter-to-microliter volumes of distinct receptor molecules are attached to individual metasurface biosensor pixels and flow over a single biological sample for analysis. In the reverse case, one step is bulk functionalization of the surface with a single probe molecule, and deposition of picoliter to microliter volumes of distinct samples onto an exemplary chip for multiplexed detection of a single biomarker.

In one embodiment, acoustic bioprinting (acoustic droplet ejection) is utilized to pattern the probe molecules. Acoustic printing works by using ultrasonic waves to eject a droplet from a free surface of fluid. A radio frequency (RF) burst signal is used to excite a transducer at its resonant frequency, generating ultrasonic waves that exert force on the fluid surface. When the focus of the transducer is aligned with the liquid-air interface and the intensity of the acoustic field is high enough, the generated radiation pressure is greater than the force of the surface tension, and a droplet is ejected from the fluid surface due to the Rayleigh-Taylor instability. The diameter of the ejected droplet is inversely proportional to the frequency of the transducer, with 5 MHz and 300 MHz ultrasonic waves generating droplet diameters of 300 μm and 5 μm, respectively. The size, speed, and directionality of the ejected droplets are completely controlled by the sound waves without the need for a physical nozzle, like other commercial piezo or thermal inkjet printers. As a nozzle-less technology, acoustic droplet ejection has an unparalleled advantage in handling biological samples: in particular, it eliminates clogging, sample contamination, and compromised cell viability or biomarker structure due to shear forces from the nozzle. Furthermore, acoustic droplet ejection (ADE) allows for high throughput droplet generation, processing fluids at rates of up to 25 kHz for a single ejector head. Furthermore, as this platform relies on acoustic waves, these waves can propagate through a matched coupling media with minimal loss of acoustic energy while avoiding sample contamination and maintaining sterility.

According to another exemplary aspects of the present disclosure, the small droplet size (picoliters to microliters) generated using acoustic bioprinting allows for matching of the surface functionalization resolution with the spatial resolution and patterning of the exemplary pixel arrays such that individual pixels can be modified to detect distinct biomarkers from neighboring elements or with distinct patient samples for analysis.

It is noted that such multiplexed metasurface(s) can be functionalized using any droplet generation or chemical transfer method that allows for surface functionalization at the resolution of the exemplary patterned metasurface. Other embodiments include, but are not limited to, inkjet printing, nanocontact print stamping, microcontact printing, and pipetting.

One such example is shown in FIG. 7B where bioprinting provides for such functionalization (e.g., using a 1 millimeter scale) via accurately placed droplets containing different analytes of physical dimensions ˜10-100 μm. In certain experimental examples, bioprinting of different chemistries near the physical dimensions of the individual photonic resonator devices (˜10s of microns) allows for multiplexed biomolecular detection.

In certain further experiments involving metasurface functionalization for nucleic acid detection, for surface functionalization, complementary nucleic acid sequences may be used as probe molecules. After functionalization with MBS, thiolated nucleic acid probes with complementary sequences to target molecules are attached to the surface. Probe concentration and surface densities can be tuned for highest efficiency hybridization with target nucleic acid strands by diluting the initial silane self-assembled monolayers with 10 PTMS (trimethoxy (propyl) silane). This well-studied surface functionalization approach via silanization has already been validated in connection with the present disclosure for reproducible and controllable oligonucleotide attachment.

After completion of bulk functionalization with an example silane molecule and MBS, one such manufacture methodology then attaches the probe molecules onto the surface. In one embodiment, the surface is functionalized with a monolayer of zwitterionic, polyethylene glycol (PEG)-lyated matrix optimized to minimize nonspecific adsorption. This matrix is composed of an optimized ratio of two molecules. 2-{2-[2-(1-mercaptoundec-11-yloxy)-ethoxy]-ethoxy}-ethoxy nitrilotriacetic acid (HS-C11-(EG) 3-NTA) and (2-{12-(2-[1]-mercapto-undecyloxy)-ethoxy]-ethoxy)-ethoxy]-ethoxy)-dimethylammonio)acetate, the first of which will eventually bind to the antibody of interest while the second increases the density of the monolayer. Subsequent incubation with nickel chloride salt binds to the NTA molecule. This Ni(II)-NTA complex then enabled binding of the RBD region of the SARS-CoV-2 spike protein to the exemplary metasurface. The spike protein has been modified with a polyhistidine-tag, increasing the affinity of the spike protein for metal ions, and thereby increasing its bonding affinity with the Ni(II)-NTA complex of the exemplary monolayer. Importantly, this functionalization orients the antibody recognition site, allowing for increased likelihood of bonding with the primary antibody. In another embodiment, the surface is functionalized with a thiolated biotin polyethylene glycol (PEG), which is subsequently incubated with streptavidin protein, which allows the final probe molecule to be linked with the self-assembled monolayer. After incubation with the streptavidin molecule, the chips are incubated with the probe molecules that have been biotinylated or modified in a way that that the biotin molecule is covalently attached to the proteins, allowing them to specifically bind with the streptavidin-terminated surface monolayer. Upon completion of surface functionalization to build the self-assembled monolayer with probe molecules, the chip is placed in a sealed holder, for example a PDMS fluid cell, that allows for the introduction of patient liquid samples (e.g. blood, serum, plasma, urine, saliva, etc.) onto the chip without contamination, allowing for simultaneous optical interrogation and read-out, and for monitoring the kinetic binding response of the system.

In connection with certain examples and specific applications, aspects of the present disclosure have application in clinical and research settings for diagnosing and monitoring human, animal, and ecosystem health. For example, detection of many genetic mutations or protein signatures in parallel is important for treatment of various cancers, cardiovascular diseases, and neurological disorders. Identification of both metabolites and environmental DNA and proteins is often considered important for monitoring ocean biodiversity and spread of marine toxins. According to the present disclosure, certain platform-based aspects allow for the study of multi-biomarker associations for better understanding of diseases previously unavailable due to limitations of measuring multiple analytes in a single sample. In an exemplary clinical setting, other aspects of the present disclosure provide a rapid and low cost device to produce longitudinal data of biomarker or metabolite levels related to a treatment plan and allow for quick adjustment or optimization of patient treatment plans.

In certain contexts, advantages and improvements over existing methods, devices or materials include massive multiplexing capabilities with sensing elements patterned at densities exceeding 1,000,000 features per square cm, and the potential to analyze multiple classes of biomarkers on a single chip (such as both nucleic acids and proteins) for more accurate or earlier diagnoses. There is also the potential to screen multiple samples for similar biomarkers on a single chip, such as screening patients for viral infection at population scale, which can perform rapid multiplexed gene and protein detection with much higher throughput than PCR, ELISA, mass spec and other methods. Metasurface elements exhibit sharp scattering spectra that sensitively change when a target biomarker binds to the silicon surface, negating the need for optical tagging processes that require additional processing and expensive reagents. Minimal sample processing, since no amplification or tagging is necessary, results in minutes rather than hours to days compared to PCR, NGS, or fluorescence based protein arrays. Rapid signal read outs with binding signals are detected within minutes (e.g., less than several minutes or in some cases ten minutes).

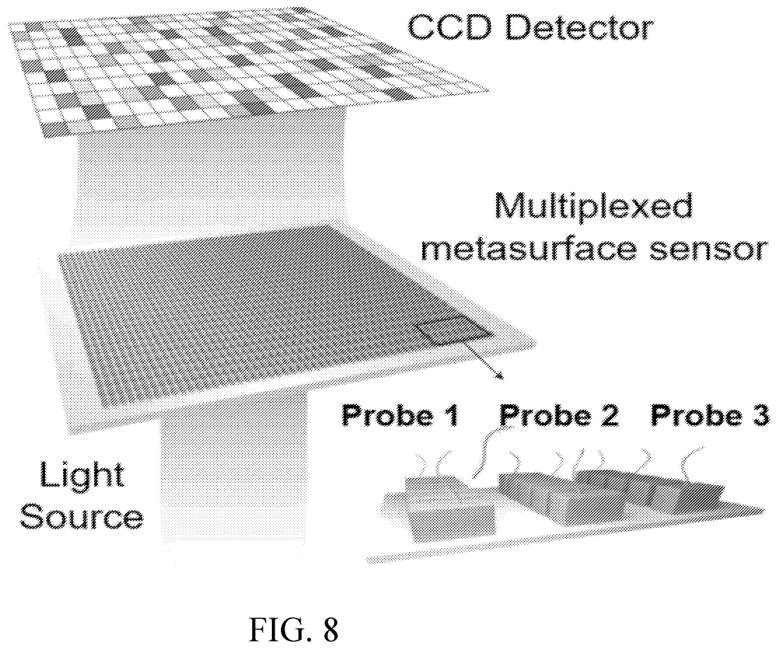

FIG. 8 is a perspective view of an example system, also according to the present disclosure and consistent with aspects of the foregoing discussion. As depicted and discussed above, the system may include a light source (e.g., laser or LED) illuminating the multiplexed metasurface sensor (e.g., from the bottom), with the sensor being functionalized to attach distinct receptor or probe molecules to individual metasurface biosensor pixels, and a CCD detector capturing light for an optical readout after being manipulated (e.g., transmitted or reflected transmitted light) in response to the functionalized sensor shown with a blowup of exemplary probes 1, 2 and 3.

It is recognized and appreciated that as specific examples, the above-characterized figures and discussion are provided to help illustrate certain aspects (and advantages in some instances) which may be used in the manufacture of such structures and devices. These structures and devices include the exemplary structures and devices described in connection with each of the figures as well as other devices, as each such described embodiment has one or more related aspects which may be modified and/or combined with the other such devices and examples as described hereinabove may also be found in the Appendices of the above-referenced Provisional Application, which correspond to U.S. Pat. No. 11,391,866 and to PCT Patent Application WO2022076832 (each incorporated by reference for subject matter overlapping in terms of design concepts such as material types, dimensions and operation of the optics elements, GMR discussion and high-Q responses and ranges characterizing GMR of a certain high-Q).

The skilled artisan would also recognize various terminology as used in the present disclosure by way of their plain meaning. As examples, the Specification may describe and/or illustrates aspects useful for implementing the examples by way of various electronic devices, optics elements, material layers, circuits, etc., which may be illustrated as or using terms such as layers, blocks, modules, device, system, unit, controller, and/or other circuit-type depictions. Such materials may include semiconductor and/or semiconductive materials, and optics, circuit elements and/or related circuitry may be used together with other elements to exemplify how certain examples may be carried out in the form or structures, steps, functions, operations, activities, etc. It would also be appreciated that terms to exemplify orientation, such as upper/lower, left/right, top/bottom and above/below, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the true spirit and scope of various aspects of the disclosure, including aspects set forth in the claims.

What is claimed:

1. An apparatus comprising:
a guided-mode resonance metasurface pixel (“GMR pixel”) having a cavity section to support GMR at a certain Q and having optics at each end of the GMR pixel, to contain light and mitigate energy losses due to scattering of light, in response to light being directed towards the GMR pixel of the metasurface sensor.

2. The apparatus of claim 1, wherein a surface of the GMR pixel is functionalized for attachment of a distinct set of one or more respective receptors and/or probe molecules, wherein in response to light being directed towards a sample and the functionalized GMR pixel, the functionalized GMR pixel is to selectively attach to a distinct type of molecular structures, among a plurality of other types molecular structures, in the biological sample.

3. The apparatus of claim 1, wherein the cavity section has multiple nanoblocks, and wherein blocks of the GMR pixel are to react to light directed towards a surface of the GMR pixel, and to cause formation of a photonic bandgap, where no photonic modes with frequencies within the bandgap are allowed to propagate, and wherein frequencies corresponding to the edge of the bandgap are tuned based on the dimensions of the nanoblocks.

4. The apparatus of claim 1, wherein the optics include, at each end of the GMR pixel and on opposing sides of the cavity section, a first set of one or more nanoblocks and a second set of one more nanoblocks.

5. The apparatus of claim 1, wherein the GMR pixel is one of a plurality of similarly-constructed the GMR pixels, each having a cavity section to support GMR at the certain Q and having optics at each end of the GMR pixel, to contain light and optimally mitigate energy losses due to scattering of light, in response to light being directed towards the GMR pixel of the metasurface sensor, and wherein the apparatus further include logic circuitry to selectively access, and discerning responsiveness of, different ones of the plurality of similarly-constructed the GMR pixels.

6. The apparatus of claim 1, wherein a surface of one of the similarly-constructed GMR pixels is functionalized for attachment of a distinct set of one or more respective receptors and/or probe molecules, and a surface of another one of the similarly-constructed GMR pixels is functionalized for attachment of a different distinct set of one or more respective receptors and/or probe molecules, with the respective surfaces being collectively functionalized to distinguish between different types of biological molecules in the biological sample.

7. The apparatus of claim 1, wherein the optics function or act as photonic mirrors by containing a resonant mode.

8. The apparatus of claim 7, wherein the cavity section has multiple nanoblocks of different dimensions, one of the different dimensions being distinguishable from another of the different dimensions in terms of nanoblock length.

9. A method comprising:
directing light towards a guided-mode resonance metasurface pixel (“GMR pixel”) of a metasurface sensor, wherein the GMR pixel has a cavity section to support GMR at a certain Q; and
containing light and mitigating energy losses due to scattering of light, in response to the light being directed towards the GMR pixel, via optics at each end of the GMR pixel.

10. The method of claim 9, wherein the cavity section has multiple nanoblocks to support GMR at a certain high-Q characterized as being greater than 1000.

11. The method of claim 9, wherein the cavity section is to support GMR at a certain high-Q characterized as being greater than 10.

12. The method of claim 9, wherein the cavity section has multiple nanoblocks of different lengths which are dimensioned such that the respective ends of the multiple nanoblocks collectively align to form a tapered dimension of at least a portion of the cavity section between the optics ends.

13. An apparatus comprising:
a functionalized metasurface sensor including an array of guided-mode resonance metasurface biosensor pixels (“GMR pixels”), wherein each of the GMR pixels has a cavity section to support GMR at a certain Q and is functionalized for attachment of a distinct receptor or probe molecules;
optics, coupled at respective ends of each of the GMR pixels, to contain light and optimally mitigate energy losses due to scattering of light, in response to light being directed towards the metasurface sensor; and
light-responsive and data-processing circuitry, responsive to the directed light manipulated by a biological sample at respective ones of the distinct receptor or probe molecules, to distinguish between different types of biological molecules in the biological sample.

14. The apparatus of claim 13, wherein at least one of the GMR pixels has a material capable of being energized by the directed light and has at least one section with a length corresponding to an illumination wavelength energy that is below a band gap of the material.

15. The apparatus of claim 13, wherein at least one of the GMR pixels has a cavity section to support GMR at a certain high-Q characterized as being greater than several thousand.

16. The apparatus of claim 13, wherein the optics are to act as photonics mirrors, and wherein the different types of biological molecules refer to or include one or more of: nucleic acids, proteins, pathogens, and small molecules.

17. The apparatus of claim 13, wherein the functionalized metasurface sensor, the optics and the light-responsive and data-processing circuitry are configured cooperatively to enable detection of multiple distinct DNA and RNA sequences, as well as different proteins, on a single chip and from a single sample, without relying on recognition any fluorescent or optical tagging.

18. The apparatus of claim 13, further including the distinct receptor or probe molecules for attachment to respective ones of the metasurface biosensor pixels.

19. The apparatus of claim 13, wherein respective ones of the metasurface biosensor pixels are bio printed.

20. The apparatus of claim 13, wherein the functionalized metasurface sensor, the optics and the light-responsive and data-processing circuitry are configured cooperatively to provide a nanophotonic waveguide device, with each of the GMR pixels acting as a waveguide that is prone to redirect certain of the light in the form of radiation losses, and wherein the optics causes a reduction of Q factors associated with photons scattering from said at least one of the GMR pixels.

21. A method comprising:
functionalizing a metasurface sensor including an array of guided-mode resonance metasurface biosensor pixels (“GMR pixels”), by attaching a distinct receptor or probe molecules to respective ones of the GMR pixels; and
with each of a plurality of the GMR pixels including optics secured at respective ends of the GMR pixel and including a cavity section to support GMR at a certain Q, directing light towards the metasurface sensor and containing light and thereby mitigating energy losses due to scattering of light, and
using circuitry to respond to the directed light, after being manipulated by a biological sample at respective ones of the GMR pixels, to distinguish different types of biological molecules in the biological sample.

22. The method of claim 21, further including distinguishing the different types of biological molecules in the biological sample without relying on recognition of any tagged target molecules.

23. The method of claim 21,
further including using the circuitry to direct the light towards the metasurface sensor, wherein the circuitry includes a CCD and a logic circuit to respond to the directed light.

24. The method of claim 21, wherein the distinct receptor or probe molecules to respective ones of the GMR pixels are patterned through acoustic droplet ejection.

25. The method of claim 21, further including customizing or matching surface functionalization resolution of individual ones of the GMR pixels to detect distinct biomarkers relative to neighboring ones of the GMR pixels or certain distinct target species of samples for analysis.

26. The method of claim 21, wherein the distinct receptor or probe molecules are attached to surfaces of the GMR pixels by applying a material to minimize nonspecific adsorption for binding to an antibody of interest.

27. The method of claim 21, wherein the GMR pixels are functionalized on a single chip to provide molecular binding with two or more classes of biomarkers selected from among one or more of the following: nucleic acids, proteins, and certain substances indicative of disease, infection, and environmental exposure, wherein the distinguishing is based on molecular binding being detected through changes in scattered light intensity from individual ones of the GMR pixels.

28. The method of claim 21, wherein said distinguishing is based on manifestations of sharp scattering spectra, responsive to the directed light, that sensitively change in response to a target biomarker binding to one or more surfaces of respective GMR pixels.
